(19) United States
(12) Patent Application Publication
(10) Pub. No.: US 2024/0241052 A1
(43) Pub. Date: Jul. 18, 2024

(54) BIOSENSOR IMPLEMENTING FP-WA COUPLING MODE, PREPARATION METHOD THEREFOR, AND USE THEREOF

(71) Applicant: SUN YAT-SEN UNIVERSITY, Haizhu District Guangzhou (CN)

(72) Inventors: Chongjun JIN, Haizhu District Guangzhou (CN); Yang SHEN, Haizhu District Guangzhou (CN); Kai HE, Haizhu District Guangzhou (CN); Shan XING, Haizhu District Guangzhou (CN)

(21) Appl. No.: 18/560,188

(22) PCT Filed: Jun. 9, 2021
(86) PCT No.: PCT/CN2021/099061

§ 371 Date: Nov. 10, 2023

(30) Foreign Application Priority Data
May 11, 2021 (CN) 202110510078.8

(51) Int. Cl. G01N 21/552; G01N 21/01; G01N 33/543; G01N 33/68

(52) U.S. Cl. CPC G01N 21/554; G01N 21/01; G01N 33/54373; G01N 33/689; G01N 2021/0112; G01N 2333/471; G01N 2800/385

(57) ABSTRACT

A biosensor for implementing an FP-WA coupled mode includes a dielectric layer and a metal layer. The dielectric layer includes a plurality of dielectric grooves, and the plurality of dielectric grooves are periodically distributed at equal intervals. An opening width of the dielectric groove gradually decreases in a direction from a groove opening to a groove bottom. The metal layer is disposed on the dielectric layer and includes metal grooves that are in one-to-one correspondence with the dielectric grooves.

### CROSS-REFERENCE TO RELATED APPLICATIONS

This application is an U.S. national stage application under 35 U.S.C. § 371 based upon international patent application No. PCT/CN2021/099061 filed on Jun. 9, 2021, which claims priority to Chinese patent application No. 202110510078.8 filed on May 11, 2021. The contents of the above-identified applications are hereby incorporated herein in their entireties by reference.

### TECHNICAL FIELD

The present disclosure relates to the technical field of sensors, and in particular to a biosensor for implementing a Fabry-Péot and Wood's anomaly (FP-WA) coupled mode, a method for preparing the same, and use thereof.

### BACKGROUND

As a global medical model changes from “centralization” to “decentralization” and digital telemedicine becomes popular, application scenarios for biomedical testing have been gradually expanded from large hospitals to communities, transportation hubs and households. Traditionally, the biomedical testing is mainly to test collected samples including blood, urine, or genetic samples in an analytical laboratory instead of on site. Nowadays, a point-of-care testing (POCT) apparatus based on a biosensor has been used to analyze samples immediately on a sampling site, thereby simplifying complicated processing procedures for sample testing in the laboratory and rapidly obtaining testing results.

In this trend, high sensitivity, low cost, rapidity, and portability has become the goal of new generation biosensors. Surface plasmon resonance (SPR) sensing, as an emerging biomolecule sensing technology in the early 1990s, can provide a biomedical test solution which is sensitive, simple, rapid, and integrated. The SPR sensing technology mainly includes two types, i.e., traditional propagating surface plasmon resonance (PSPR) and localized surface plasmon resonance (LSPR). Despite having desirable sensitivity, the PSPR requires complex apparatus, and is expensive and difficult to change from “centralization” to “decentralization”. The LSPR, despite having a simple structure, has resonance peaks with a large linewidth and a low quality factor.

### SUMMARY

In view of the above, there is a need to provide a biosensor for implementing a FP-WA coupled mode that has a simple structure, a small resonance peak linewidth and a high quality factor, a method for preparing the same, and use thereof.

The present disclosure adopts technical solutions as below.

A biosensor for implementing an FP-WA coupled mode includes a dielectric layer and a metal layer. The dielectric layer includes a plurality of dielectric grooves, and the plurality of dielectric grooves are periodically distributed at equal intervals. An opening width of the dielectric groove gradually decreases in a direction from a groove opening to a groove bottom. The metal layer is disposed on the dielectric layer and includes metal grooves that are in one-to-one correspondence with the dielectric grooves.

A period of the metal groove satisfies formula (1):

$${λ}_{W A} = \frac{a}{i} \sqrt{{ε}_{d}} . \qquad Formula ( 1 )$$

A depth of the metal groove, a width of the groove opening of the metal groove, and a width of the groove bottom of the metal groove satisfy formula (2):

$$h = \frac{2 π m - {φ}_{r}}{2} \cdot \frac{{w}_{0} - {w}_{1}}{k} \{ \frac{1}{- {w}_{1} \sqrt{1 + \frac{A}{{w}_{1}}} + {w}_{0} \sqrt{1 + \frac{A}{{w}_{0}}} + \frac{A}{2} ln [ ( \frac{\sqrt{1 + \frac{A}{{w}_{1}}} - 1}{\sqrt{1 + \frac{A}{{w}_{1}}} + 1} ) ( \frac{\sqrt{1 + \frac{A}{{w}_{0}}} + 1}{\sqrt{1 + \frac{A}{{w}_{0}}} - 1} ) ]} \} ; \qquad Formula ( 2 )$$

$$wherein A = \frac{2 \sqrt{{ε}_{d} ( {ε}_{d} - {ε}_{m} )}}{- {ε}_{m} k} , k = \frac{2 π \sqrt{{ε}_{d}}}{{λ}_{WG}} ;$$

and

a represents the period of the metal groove, h represents the depth of the metal groove, w₀ represents the width of the groove opening of the metal groove, w₁ represents the width of the groove bottom of the metal groove, i represents an order of a WA mode, λ_{WA} represents a resonance wavelength of the WA mode, m represents an order of an FP mode, λ_{WG} represents a resonance wavelength of the FP mode, k represents a resonance wave number of the FP mode, φᵣ represents a sum of reflection phases of the FP mode at the groove opening of the metal groove and the groove bottom of the metal groove, ε_{d} represents a dielectric constant of an environment where the biosensor is located, and εₘ represents a dielectric constant of the metal layer.

In an embodiment, the period of the metal groove is from 600 nm to 1500 nm. The depth of the metal groove is from 300 nm to 800 nm. The width of the groove opening of the metal groove is from 400 nm to 600 nm. The width of the groove bottom of the metal groove is from 200 nm to 400 nm.

In an embodiment, a resonance linewidth of the biosensor is from 3 nm to 9 nm.

In an embodiment, a thickness of the metal layer is from 200 nm to 500 nm.

In an embodiment, a root mean square of surface roughness of the metal layer is from 0.2 nm to 1.9 nm.

In an embodiment, the plurality of dielectric grooves are distributed in a plurality of rows and/or in a plurality of columns, the rows are parallel to each other, the columns are parallel to each other, and the rows are perpendicular to the columns.

In an embodiment, the dielectric grooves in each row are in communication with each other to form a communicated groove with a uniform width. Alternatively, the dielectric grooves in each column are in communication with each other to form a communicated groove with a uniform width.

In an embodiment, the biosensor further includes a substrate, and the substrate is disposed on a surface of the dielectric layer away from the metal layer.

In an embodiment, the metal layer is at least one of a gold layer, a platinum layer, or a silver layer.

In an embodiment, the dielectric layer is a thermosetting epoxy resin dielectric layer, or a photocurable epoxy resin dielectric layer.

A method for preparing the biosensor in any embodiment as described above includes the following steps:
forming auxiliary protrusions that are distributed periodically at equal intervals on a template;
forming a metal layer on a surface of the template, the metal layer including metal grooves that are in one-to-one correspondence with the auxiliary protrusions;
forming a dielectric layer on the metal layer; and
separating the metal layer from the template.

In an embodiment, the template is a silicon wafer.

Use of the biosensor in any embodiment as described above in the measurement of a parameter of interest of a target for a non-disease diagnosis purpose.

In an embodiment, the use includes the following steps:
radiating incident light onto a surface of the metal layer in a direction perpendicular to the groove bottom of the metal groove, and measuring an initial optical parameter of reflected light;
adding dropwise different target solutions with known parameters of interest onto the surface of the metal layer respectively, and measuring standard optical parameters of reflected light corresponding to the target solutions;
calculating difference values between the standard optical parameters corresponding to the different target solutions with known parameters of interest and the initial optical parameter respectively, and obtaining a change relation between the optical parameter and the parameter of interest of the target solution based on the difference values and the corresponding known parameters of interest; and
adding dropwise a solution to be tested onto the surface of the metal layer, measuring a sample optical parameter of reflected light corresponding to the solution to be tested, calculating a difference value between the sample optical parameter and the initial optical parameter, and obtaining a parameter of interest of the solution to be tested according to the difference value and the change relation.

In an embodiment, the plurality of dielectric grooves are distributed in a plurality of rows and/or in a plurality of columns, the rows are parallel to each other, the columns are parallel to each other, and the rows are perpendicular to the columns. A polarization direction of the incident light is perpendicular to the row direction, or a polarization direction of the incident light is perpendicular to the column direction.

In an embodiment, the dielectric grooves in each row are in communication with each other to form a communicated groove with a uniform width. Alternatively, the dielectric grooves in each column are in communication with each other to form a communicated groove with a uniform width. The polarization direction of the incident light is perpendicular to an extending direction of the communicated groove.

In an embodiment, the target is a salt, an organic solvent, an antigen, an antibody, deoxyribonucleic acid (DNA) or ribonucleic acid (RNA).

According to the biosensor described above, the structure is set to form a plurality of metal grooves distributed periodically at equal intervals on the dielectric layer. Each metal groove is equivalent to a resonator and supports the Fabry-Péot cavity mode (i.e., FP mode) with a wide linewidth, while periodicity of the metal grooves induces the Wood's anomaly mode (i.e., WA mode). According to a relation between structural parameters and the resonance wavelength in Formula (1) and Formula (2), the period of the metal groove, the depth of the metal groove, the width of the groove opening of the metal groove, and the width of the groove bottom of the metal groove are set, such that wavelengths of the FP mode and the WA mode get close to each other, to obtain a coupled mode that has an ultra-narrow linewidth and an ultra-deep reflection valley (i.e., the FP-WA coupled mode), thereby further obtaining the biosensor with the small resonance peak linewidth and the high quality factor. Besides, the biosensor has a simple structure, and the FP-WA coupled mode can be excited through vertical incidence, thus avoiding the high-precision angle adjustment assembly required by the biosensor which is based on oblique incidence excitation, facilitating integration of devices to a greater extent, being beneficial to transportation and application, and effectively facilitating the biological testing to change from “centralization” to “decentralization”.

According to the method for preparing the biosensor, periodical auxiliary protrusions are first formed on a template, a metal layer is formed on surfaces of the auxiliary protrusions to form metal grooves, then a dielectric layer is formed on the surface of the metal layer, and then the metal layer is separated from the template. According to the preparation method, the metal layer can have a surface as smooth as that of the template, thereby effectively improving the smoothness of the metal layer. As a result, the linewidth of the resonance peak can be further decreased and the quality factor can be improved. In addition, using the preparation method can effectively avoid the problem that an optical response of the biosensor is affected by thickness change of the metal layer and asymmetry of the metal layer.

The use of the biosensor in the measurement of a parameter of interest of a target for a non-disease diagnosis purpose is convenient and easy to operate, does not need to rely on a detector with a high wavelength resolution, and has low detection cost.

### BRIEF DESCRIPTION OF THE DRAWINGS

FIG. 1 is a schematic structural diagram of a biosensor according to an embodiment of the present disclosure.

FIG. 2 shows size symbols of a metal groove in the biosensor corresponding to FIG. 1.

FIG. 3 is a schematic structural diagram of a mask template in a preparation process of the biosensor corresponding to FIG. 1.

FIG. 4 is a schematic structural diagram of auxiliary protrusions in the preparation process of the biosensor corresponding to FIG. 1.

FIG. 5 is a schematic structural diagram showing formation of a metal layer in the preparation process of the biosensor corresponding to FIG. 1.

FIG. 6 is a schematic structural diagram showing formation of a dielectric layer and a substrate in the preparation process of the biosensor corresponding to FIG. 1.

FIG. 7 shows a physical picture of a biosensor according to an embodiment of the present disclosure.

FIG. 8 shows a top view under electron microscopy of periodical metal grooves in the biosensor corresponding to FIG. 1.

FIG. 9 shows a side view under electron microscopy of periodical metal grooves in the biosensor corresponding to FIG. 1.

FIG. 10 shows an atomic force micrograph of a metal layer in the biosensor corresponding to FIG. 1.

FIG. 11 shows (a) a reflection spectrum of an FP mode, (b) a reflection spectrum of a WA mode, and (c) a reflection spectrum of an FP-WA coupled mode.

FIG. 12 shows a reflection spectrum of an FP-WA coupled mode with a resonance linewidth of 2 nm.

FIG. 13 shows reflection spectra of six biosensors duplicated on a same silicon template.

FIG. 14 shows (a) reflection spectra of a gold nano groove array with different groove depths and (b) a spectrum under an optimal groove depth parameter.

FIG. 15 is a schematic diagram showing avoidance of an asymmetric effect and a thickness error of a gold layer during gold plating in a preparation method according to an embodiment of the present disclosure.

FIG. 16 shows reflection spectra of the biosensor corresponding to FIG. 1 when alpha-fetoprotein (AFP) antigen solutions with different concentrations are added dropwise.

FIG. 17 shows (a) a relation curve between a wavelength shift of the biosensor corresponding to FIG. 1 and an AFP concentration and (b) a relation curve between a relative reflectivity change of the biosensor corresponding to FIG. 1 and the AFP concentration.

FIG. 18 shows (a) reflection spectra of the biosensor corresponding to FIG. 1 in glycerol aqueous solutions with different concentrations and (b) a relation curve between a wavelength shift of the biosensor corresponding to FIG. 1 and refractive index of the glycerol aqueous solutions with different concentrations.

FIG. 19 shows reflection spectra of metal grooves with different working wavelengths.

FIG. 20 shows (a) a schematic diagram of a one-dimensional array of metal grooves and (b) a schematic diagram of a two-dimensional array of metal grooves.

100. biosensor; 101. dielectric layer; 102. metal layer; 1021. metal groove; 103. substrate; 200. template; 201. auxiliary protrusion; and 300. mask material.

### DETAILED DESCRIPTION OF THE EMBODIMENTS

To make the above objectives, features and advantages of the present disclosure clearer and more comprehensible, specific embodiments of the present disclosure will be described below in detail. In the following description, numerous specific details are set forth for the convenience of full understanding of the present disclosure. However, the present disclosure can be implemented in many other manners different from those described herein, those skilled in the art can make similar improvements without departing from the connotation of the present disclosure, and the present disclosure will not be limited to specific embodiments disclosed below.

In addition, the terms “first” and “second” are merely used for describing purposes and cannot be understood as indicating or implying relative importance, or implicitly indicating the number of indicated technical features. In view of that, the features defined with “first” and “second” can explicitly or implicitly include at least one of the features. In the description of the present disclosure, “a plurality of” means at least two, including two, three, etc., unless otherwise explicitly and specifically defined.

In the present disclosure, unless otherwise explicitly specified and defined, the terms including “mount”, “connected”, “connection”, “fix”, etc. should be understood broadly. For example, “connection” can be a fixed connection, a detachable connection or an integral connection, a mechanical connection or an electric connection, a direct connection or an indirect connection through an intermediate medium, and internal communication of two elements or interaction between the two elements, unless otherwise explicitly defined. For those skilled in the art, specific meanings of the above terms in the present disclosure can be understood according to specific circumstances.

Unless otherwise defined, all technical and scientific terms used herein have the same meanings as those commonly understood by those skilled in the technical field of the present disclosure. The terms used in the description of the present disclosure are merely for the purpose of describing particular embodiments, and are not intended to limit the present disclosure. As used herein, the term “and/or” includes any or all combinations of one or more relevant listed items.

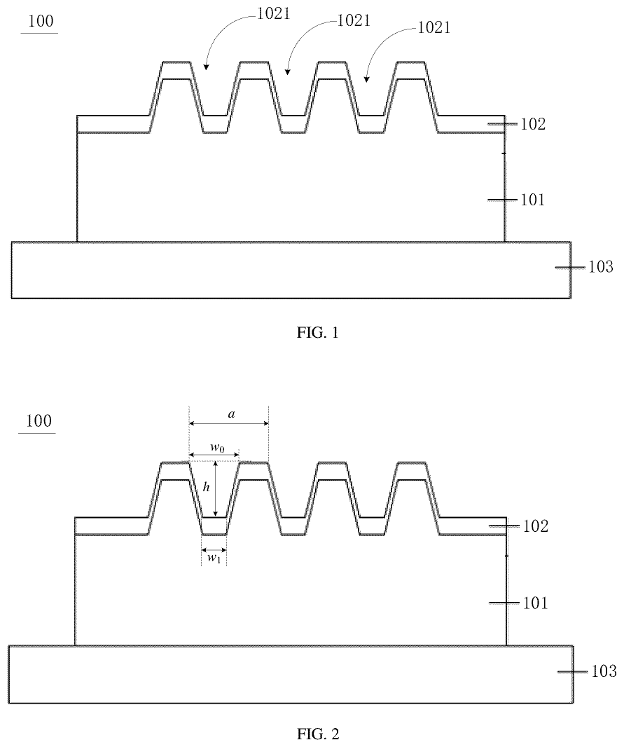

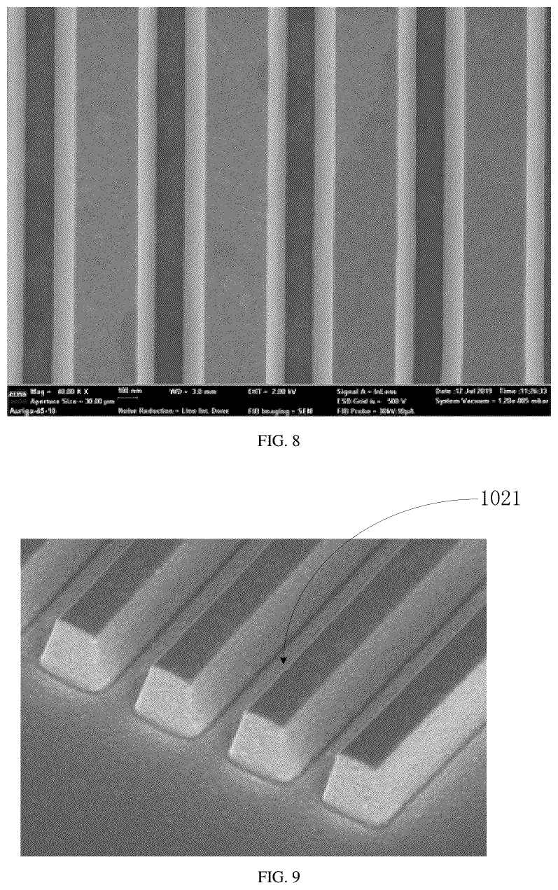

With reference to FIGS. 1, 2, 8 and 9, an embodiment of the present disclosure provides a biosensor 100 for implementing a FP-WA coupled mode. The biosensor 100 includes a dielectric layer 101 and a metal layer 102. The dielectric layer 101 includes a plurality of dielectric grooves, and the plurality of dielectric grooves are periodically distributed at equal intervals. An opening width of the dielectric groove gradually decreases in a direction from a groove opening to a groove bottom. The metal layer 102 is disposed on the dielectric layer 101 and includes metal grooves 1021 that are in one-to-one correspondence with the dielectric grooves.

A period of the metal groove 1021 satisfies formula (1):

$${λ}_{W A} = \frac{a}{i} \sqrt{{ε}_{d}} . \qquad Formula ( 1 )$$

A depth of the metal groove 1021, a width of the groove opening of the metal groove 1021, and a width of the groove bottom of the metal groove 1021 satisfy formula (2):

$$h = \frac{2 π m - {φ}_{r}}{2} \cdot \frac{{w}_{0} - {w}_{1}}{k} \{ \frac{1}{- {w}_{1} \sqrt{1 + \frac{A}{{w}_{1}}} + {w}_{0} \sqrt{1 + \frac{A}{{w}_{0}}} + \frac{A}{2} ln [ ( \frac{\sqrt{1 + \frac{A}{{w}_{1}}} - 1}{\sqrt{1 + \frac{A}{{w}_{1}}} + 1} ) ( \frac{\sqrt{1 + \frac{A}{{w}_{0}}} + 1}{\sqrt{1 + \frac{A}{{w}_{0}}} - 1} ) ]} \} ; \qquad Formula ( 2 )$$

$$wherein A = \frac{2 \sqrt{{ε}_{d} ( {ε}_{d} - {ε}_{m} )}}{- {ε}_{m} k} , k = \frac{2 π \sqrt{{ε}_{d}}}{{λ}_{WG}} ;$$

and

a represents the period of the metal groove, h represents the depth of the metal groove, w₀ represents the width of the groove opening of the metal groove, w₁ represents the width of the groove bottom of the metal groove, i represents an order of a WA mode, λ_{WA} represents a resonance wavelength of the WA mode, m represents an order of an FP mode, λ_{WG} represents a resonance wavelength of the FP mode, k represents a resonance wave number of the FP mode, φᵣ represents a sum of reflection phases of the FP mode at the groove opening of the metal groove and the groove bottom of the metal groove, ε_{d} represents a dielectric constant of an environment where the biosensor is located, and εₘ represents a dielectric constant of the metal layer.

In an embodiment, the period of the metal groove is from 600 nm to 1500 nm, the depth of the metal groove is from 300 nm to 800 nm, the width of the groove opening of the metal groove is from 400 nm to 600 nm, and the width of the groove bottom of the metal groove is from 200 nm to 400 nm. In an embodiment, the period of the metal groove may be, but is not limited to, 600 nm, 650 nm, 700 nm, 750 nm, 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, or 1500 nm. The depth of the metal groove may be, but is not limited to, 300 nm, 330 nm, 350 nm, 375 nm, 380 nm, 400 nm, 425 nm, 450 nm, 475 nm, 500 nm, 505 nm, 530 nm, 555 nm, 580 nm, 600 nm, 610 nm, 640 nm, 665 nm, 695 nm, 710 nm, 725 nm, 755 nm, 785 nm, or 800 nm. The width of the groove opening of the metal groove may be, but is not limited to, 400 nm, 420 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm. The width of the groove bottom of the metal groove may be, but is not limited to, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, or 400 nm.

In a specific embodiment, a resonance linewidth of the biosensor is from 3 nm to 9 nm. For example, the resonance linewidth of the biosensor may be 3.1 nm, 3.2 nm, 3.3 nm, 3.4 nm, 3.5 nm, 3.6 nm, 3.7 nm, 3.8 nm, 3.9 nm, 4.0 nm, 4.1 nm, 4.2 nm, 4.3 nm, 4.4 nm, 4.5 nm, 4.6 nm, 4.7 nm, 4.8 nm, 4.9 nm, 5.0 nm, 5.1 nm, 5.2 nm, 5.3 nm, 5.4 nm, 5.5 nm, 5.6 nm, 5.7 nm, 5.8 nm, 5.9 nm, 6.0 nm, 6.1 nm, 6.2 nm, 6.3 nm, 6.4 nm, 6.5 nm, 6.6 nm, 6.7 nm, 6.8 nm, 6.9 nm, 7.0 nm, 7.1 nm, 7.2 nm, 7.3 nm, 7.4 nm, 7.5 nm, 7.6 nm, 7.7 nm, 7.8 nm, 7.9 nm, 8.0 nm, 8.1 nm, 8.2 nm, 8.3 nm, 8.4 nm, 8.5 nm, 8.6 nm, 8.7 nm, 8.8 nm, 8.9 nm, or 9.0 nm.

In the biosensor according to this embodiment, the structure is set to form a plurality of metal grooves distributed periodically at equal intervals on the dielectric layer. Each metal groove is equivalent to a resonator and supports the Fabry-Péot cavity mode (i.e., FP mode) with a wide linewidth, while periodicity of the metal grooves induces the Wood's anomaly mode (i.e., WA mode). According to a relation between structural parameters and the resonance wavelength in Formula (1) and Formula (2), the period of the metal groove, the depth of the metal groove, the width of the groove opening of the metal groove, and the width of the groove bottom of the metal groove are set, such that wavelengths of the FP mode and the WA mode get close to each other, to obtain a coupled mode that has an ultra-narrow linewidth and an ultra-deep reflection valley (i.e., the FP-WA coupled mode), thereby further obtaining the biosensor with the small resonance peak linewidth and the high quality factor. Besides, the biosensor has a simple structure, and the FP-WA coupled mode can be excited through vertical incidence, thus avoiding the high-precision angle adjustment assembly required by the biosensor which is based on oblique incidence excitation, facilitating integration of devices to a greater extent, being beneficial to transportation and application, and effectively facilitating the biological testing to change from “centralization” to “decentralization”.

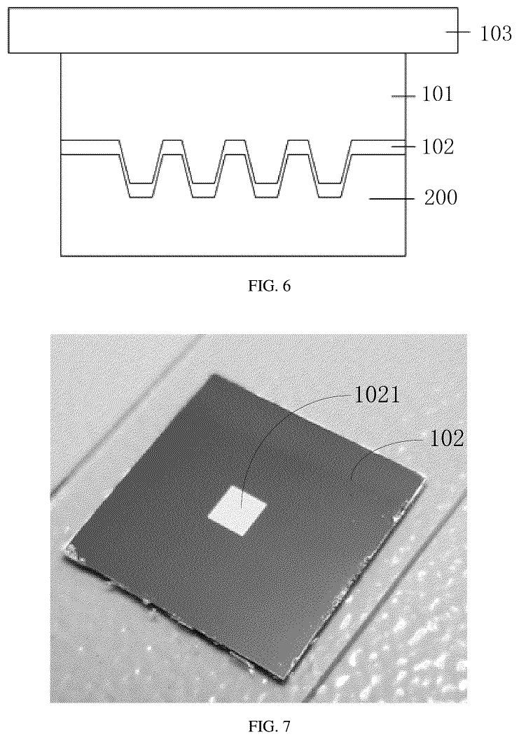

It may be understood that, as shown in FIG. 7, in actual design, a partial region of the metal layer 102 can include the metal groove 1021. Further, an area of the metal layer 102 can be greater than an area of the surface of the dielectric layer having the dielectric grooves.

It may also be understood that the metal layer covers the surface of the dielectric layer to form the corresponding metal grooves on the surfaces of the dielectric grooves. In other words, the metal layer entirely covers the dielectric grooves, such that the metal grooves cover the surfaces of the dielectric grooves. In this case, the bottom surface and side surfaces of the dielectric groove and a connection surface between two adjacent dielectric grooves are all covered by the metal layer.

When designing the biosensor, the inventors found that each metal groove in the biosensor may be regarded as one resonator, and the metal groove can support the FP mode with a wide linewidth, as shown in (a) of FIG. 11. The FP mode as a local area mode has a strong local area electric field and large radiation loss. The resonance wavelength of the PF mode can be adjusted by adjusting the depth of the metal groove, the width of the groove opening of the metal groove, and the width of the groove bottom of the metal groove. In addition, the periodicity of the metal grooves induces the WA mode. The WA mode is a propagation mode, with ultra-low radiation loss and ultra-narrow linewidth, as shown in (b) of FIG. 11. The resonance wavelength of the WA mode may be adjusted by adjusting the period of the metal groove. Based on above, according to the relation between structural parameters and the resonance wavelength in Formula (1) and Formula (2), the period of the metal groove, the depth of the metal groove, the width of the groove opening of the metal groove, and the width of the groove bottom of the metal groove are set, such that wavelengths of the FP mode and the WA mode get close to each other, to obtain a coupled mode that has an ultra-narrow linewidth and an ultra-deep reflection valley. In this case, a critical coupling condition can be achieved, such that the resonance wavelengths of the FP mode and the WA mode can get close to each other or even be identical. A radiation attenuation rate is equal to an internal attenuation rate. The FP-WA coupled mode with small resonance peak linewidth, high quality factor, high optical contrast, and strong local area field as shown in (c) of FIG. 11, is generated. Herein, the radiation attenuation rate indicates a rate at which surface plasmons of a system are converted into free-space photons, while the internal attenuation rate indicates a rate at which the surface plasmons of the system are converted into heat energy inside the system.

In a specific embodiment, a thickness of the metal layer is from 200 nm to 500 nm. If the thickness of the metal layer is too small, there may be a risk that light enters the dielectric layer through the metal layer, resulting in reduction of testing accuracy. If the thickness of the metal layer is too large, preparation cost of the sensor can be increased. Alternatively, the thickness of the metal layer may be, but is not limited to, 200 nm, 250 nm, 300 nm, 350 nm, 400 nm, 450 nm, or 500 nm. The thickness of the metal layer is preferably 300 nm.

Further, the metal layer is a stable metal layer. Further, the metal layer is at least one of a gold layer, a platinum layer, or a silver layer. It may be understood that the metal layer may be a single-layer structure of a stable metal layer, or a laminated structure composed of different stable metal layers. For example, the metal layer may be a single-layer structure of the gold layer, the platinum layer or the silver layer, or a laminated structure composed of at least two of the gold layer, the platinum layer, or the silver layer.

As a preferred range of surface roughness of the metal layer, the root mean square of the surface roughness of the metal layer is from 0.2 nm to 1.9 nm. In this case, the metal layer has a very smooth surface, thus effectively reducing scattering loss caused by surface particle effects of the metal layer, and further decreasing the resonance peak linewidth of the FP-WA coupled mode. Preferably, the root mean square of the surface roughness of the metal layer does not exceed 0.5 nm. Further, the root mean square of the surface roughness of the metal layer does not exceed preferably 0.36 nm. It may be understood that the root mean square of the surface roughness of the metal layer is from 0.2 nm to 1.9 nm. In other words, the surface of the metal layer away from the dielectric layer is of a root mean square roughness ranging from 0.2 nm to 1.9 nm. The root mean square of the surface roughness of the metal layer is set to be from 0.2 nm to 1.9 nm, such that the quality factor of the biosensor can be further improved. In a specific example, the quality factor of the biosensor may reach 285. More specifically, when the metal layer is a gold layer, the root mean square of the surface roughness of the metal layer is preferably from 0.2 nm to 0.8 nm. When the metal layer is the silver layer, the root mean square of the surface roughness of the metal layer is preferably from 0.4 nm to 1.5 nm.

It may be understood that the dielectric layer is a thermosetting epoxy resin dielectric layer, or a photocurable epoxy resin dielectric layer. Further, the photocurable epoxy resin dielectric layer is an ultraviolet cured epoxy resin dielectric layer.

In a specific example, the plurality of dielectric grooves are distributed in a plurality of rows and/or in a plurality of columns, the rows are parallel to each other, the columns are parallel to each other, and the rows are perpendicular to the columns. Specifically, the groove opening of the dielectric groove is in a square shape, and the width of the groove opening is equal to a side length of the square. In another specific example, the groove opening of the dielectric groove is in a circular shape, and the width of the groove opening is equal to a diameter of the circle.

In another specific example, the dielectric grooves in each row are in communication with each other to form a communicated groove with a uniform width. Alternatively, the dielectric grooves in each column are in communication with each other to form a communicated groove with a uniform width. In this case, the structure of the metal grooves corresponds to the structures as shown in FIGS. 1, 2, 8 and 9.

With reference to FIG. 20, when the plurality of dielectric grooves are distributed in a plurality of rows and/or in a plurality of columns, the rows are parallel to each other, the columns are parallel to each other, and the rows are perpendicular to the columns, two-dimensional array distribution of the metal grooves can be formed, as shown in (b) of FIG. 20. When the dielectric grooves in each row are in communication with each other, or when the dielectric grooves in each column are in communication with each other, one-dimensional array distribution of the metal grooves can be formed, as shown in (a) of FIG. 20.

In a specific example, the biosensor 100 further includes a substrate 103. The substrate 103 is disposed on a surface of the dielectric layer 101 away from the metal layer 102. Optionally, the substrate 103 is a glass substrate.

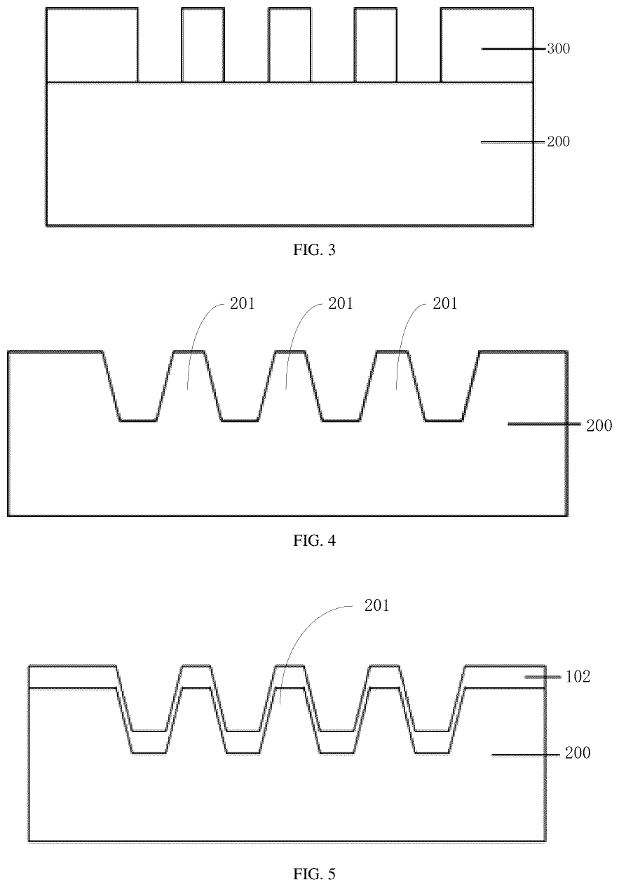

With reference to FIGS. 3 to 6, another embodiment of the present disclosure provides a method for preparing the biosensor as described above. The method includes the following steps: forming auxiliary protrusions 201 that are distributed periodically at equal intervals on a template 200; forming a metal layer 102 on a surface of the template 200, the metal layer 102 including metal grooves 1021 that are in one-to-one correspondence with the auxiliary protrusions 201; forming a dielectric layer 101 on the metal layer 102; and separating the metal layer 102 from the template. According to the preparation method, the metal layer can have a surface as smooth as that of the template, thereby effectively improving the smoothness of the metal layer. As a result, the linewidth of the resonance peak can be further decreased and the quality factor can be improved. In addition, using the preparation method can effectively avoid the problem that an optical response of the biosensor is affected by thickness change of the metal layer and asymmetry of the metal layer. Preferably, the template is a silicon wafer. The silicon wafer has better smoothness, thereby further improving the smoothness of the metal layer.

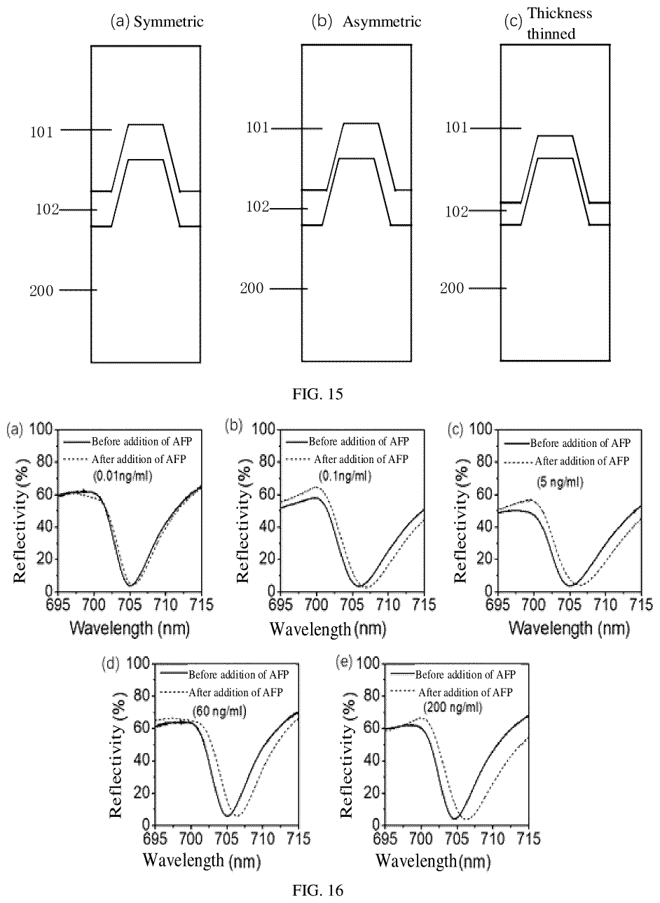

Specifically, when the biosensor is prepared by the method in this embodiment, the optical response of the metal groove is mainly determined by an interface between the silicon wafer and the metal layer. When the thickness of the metal layer is symmetrical as shown in (a) of FIG. 15, asymmetrical as shown in (b) of FIG. 15, or becomes thinner as shown in (c) of FIG. 15, the interface between the silicon wafer and the metal layer is consistent, such that the optical response of the biosensor is not affected.

Further, using the preparation method in this embodiment can obtain a metal layer with a root mean square of surface roughness from 0.2 nm to 1.9 nm, thus greatly improving the smoothness of the metal layer, and further decreasing the resonance peak linewidth of the FP-WA coupled mode. In contrast, a conventional metal film which is prepared by direct evaporation deposition or sputtering, has a root mean square of surface roughness greater than 2 nm. For example, a conventional gold film which is prepared by direct evaporation deposition or sputtering has a root mean square of surface roughness greater than 2 nm. A conventional silver film which is prepared by direct evaporation deposition or sputtering has a root mean square of surface roughness greater than 4 nm. That is, this embodiment provides a preparation method that can effectively reduce the root mean square of the surface roughness of the metal layer, and thus this preparation method can be applied to prepare a metal layer with a low surface roughness.

In a specific example, forming periodical auxiliary protrusions 201 on a template includes: covering a surface of the template with a mask material; subjecting the mask material to exposure and development to form a mask material 300 matching the auxiliary protrusions 201 on the surface of the template, thereby obtaining a mask template as shown in FIG. 3; etching the mask template to form the auxiliary protrusions 201; and removing the mask material on the mask template.

It may be understood that the mask material may be, but not limited to, polymethyl methacrylate. In an actual operation process, the surface of the silicon template is spin-coated with a polymethyl methacrylate film. Preferably, the thickness of the polymethyl methacrylate film is controlled to be from 850 nm to 950 nm, more preferably 900 nm.

After covered on the surface of the template, the mask material is exposed and developed to obtain the mask template, as shown in FIG. 3. The mask template is etched, and then the remaining mask material on the mask template is removed to obtain the template including the periodical auxiliary protrusions 201, as shown in FIG. 4. After the template as shown in FIG. 4 is obtained, a metal layer is formed on the template, thereby obtaining the template including the metal layer as shown in FIG. 5. Then, a dielectric layer is formed on the template including the metal layer. Further, a substrate may be formed on the surface of the dielectric layer to obtain the structure as shown in FIG. 6. It may be understood that the exposure, development, and etching can be performed using a standard electron beam lithography process. The metal layer can be formed on the template through a metal deposition process, and the thickness of the metal layer is preferably controlled to be from 200 nm to 500 nm. After the template including the metal layer as shown in FIG. 5 is obtained, the metal layer is separated from the template, thereby obtaining a metal layer with a smooth surface and periodical metal grooves.

Further, the forming the dielectric layer on the metal layer includes the following steps: transferring thermosetting epoxy resin or photocurable epoxy resin onto the surface of the metal layer, and then curing for shaping. Further, before curing for shaping, a substrate is disposed on the surface of the dielectric layer to promote the shaping of the dielectric layer. Alternatively, the substrate is glass.

In a specific example, after separating the metal layer from the template, the method further includes a step of: cleaning the template. The template is cleaned to remove residual dielectric layer material and residual metal layer material on the surface of the template, so that the template can be reused. Specifically, cleaning the template includes a step of: cleaning the template with acetone, a mixture solution of iodine/potassium iodide, and ethanol sequentially. Further, a mass ratio of iodine, potassium iodide, and water in the mixture solution of iodine/potassium iodide is 3:(8 to 12):(135 to 145). Further, a mass ratio of iodine, potassium iodide, and water in the mixture solution of iodine/potassium iodide is 3:10:140.

Another embodiment of the present disclosure provides use of the biosensor in the measurement of a parameter of interest of a target. Specifically, the use includes the following steps:
radiating incident light onto a surface of the metal layer in a direction perpendicular to the groove bottom of the metal groove, and measuring an initial optical parameter of reflected light;
adding dropwise different target solutions with known parameters of interest onto the surface of the metal layer respectively, and measuring standard optical parameters of reflected light corresponding to the target solutions;
calculating difference values between the standard optical parameters corresponding to the different target solutions with known parameters of interest and the initial optical parameter respectively, and obtaining a change relation between the optical parameter and the parameter of interest of the target solution based on the difference values and the corresponding known parameters of interest; and
adding dropwise a solution to be tested onto the surface of the metal layer, measuring a sample optical parameter of reflected light corresponding to the solution to be tested, calculating a difference value between the sample optical parameter and the initial optical parameter, and obtaining a parameter of interest of the solution to be tested according to the difference value and the change relation.

Further, another embodiment of the present disclosure provides use of the biosensor in the measurement of a parameter of interest of a target for a non-disease diagnosis purpose. Specifically, the use includes the following steps:
radiating incident light onto a surface of the metal layer in a direction perpendicular to the groove bottom of the metal groove, and measuring an initial optical parameter of reflected light;
adding dropwise different target solutions with known parameters of interest onto the surface of the metal layer respectively, and measuring standard optical parameters of reflected light corresponding to the target solutions;
calculating difference values between the standard optical parameters corresponding to the different target solutions with known parameters of interest and the initial optical parameter respectively, and obtaining a change relation between the optical parameter and the parameter of interest of the target solution based on the difference values and the corresponding known parameters of interest; and
adding dropwise a solution to be tested onto the surface of the metal layer, measuring a sample optical parameter of reflected light corresponding to the solution to be tested, calculating a difference value between the sample optical parameter and the initial optical parameter, and obtaining a parameter of interest of the solution to be tested according to the difference value and the change relation.

Specifically, the target is a salt, an organic solvent, an antigen, an antibody, deoxyribonucleic acid (DNA) or ribonucleic acid (RNA). The parameter of interest is a parameter that may be reflected by an optical parameter, such as a concentration, a refractive index, etc.

In a specific example, use of the biosensor in the measurement of a concentration of a biological target for a non-disease diagnosis purpose is provided. Specifically, the use includes the following steps:
radiating incident light onto a surface of the metal layer in a direction perpendicular to the groove bottom of the metal groove, and measuring an initial optical parameter of reflected light;
adding dropwise different biological target solutions with known concentrations onto the surface of the metal layer respectively, and measuring standard optical parameters of reflected light corresponding to the different biological target solutions with known concentrations;
calculating difference values between the standard optical parameters corresponding to the different biological target solutions with known concentrations and the initial optical parameter respectively, and obtaining a change relation between the optical parameter and the concentration based on the difference values and the corresponding concentrations; and
adding dropwise a solution to be tested onto the surface of the metal layer, measuring an optical parameter of reflected light corresponding to the solution to be tested, calculating a difference value between the optical parameter and the initial optical parameter, and obtaining a concentration of the solution to be tested according to the difference value and the change relation.

Further, the biological target is an antigen, an antibody, DNA or RNA.

It may be understood that the optical parameter is at least one of resonance wavelength, reflectivity, or reflected light intensity. The resonance wavelength is a wavelength at the lowest point of the reflection valley of the biosensor. The reflectivity refers to a ratio of the reflected light intensity of the biosensor to the reflected light intensity of a silver mirror (100% reflection calibration). It may also be understood that calculating a difference value between optical parameters refers to that a difference value between a same type of optical parameters is calculated.

As a specific example of the above use, the use is a use of the biosensor in the measurement of a concentration of an antigen. Specifically, the use includes the following steps:
adding dropwise an antibody solution onto a surface of the metal layer of the biosensor, and immobilizing the antibody;
radiating incident light onto the surface of the metal layer in a direction perpendicular to the groove bottom of the metal groove, and measuring an initial optical parameter of reflected light;
adding dropwise different antigen solutions with known concentrations onto the surface of the metal layer respectively, allowing the antigen to be specifically bound to the antibody, and respectively measuring test optical parameters of reflected light corresponding to the different antigen solutions with known concentrations;
calculating difference values between the test optical parameters corresponding to the different antigen solutions with known concentrations and the initial optical parameter respectively, and obtaining a change curve between the optical parameter and the concentration of the antigen solution based on the difference values and the corresponding known concentrations of the antigen solutions; and
adding dropwise an antigen solution to be tested onto the surface of the metal layer, measuring an optical parameter of reflected light corresponding to the antigen solution to be tested, calculating a difference value between the optical parameter and the initial optical parameter, and obtaining an antigen concentration of the antigen solution to be tested according to the difference value and the change curve.

In a specific example, the immobilizing the antibody includes steps of: incubating at a temperature of 37° C. after addition of the antibody solution, washing three times with a phosphate buffer solution and deionized water sequentially, and drying with a nitrogen gas. The allowing the antigen to be specifically bound to the antibody includes steps of: incubating at a temperature of 37° C. after addition of the antigen solution, washing three times with a phosphate buffer solution and deionized water sequentially, and drying with a nitrogen gas.

In another specific example, before adding dropwise an antibody solution onto a surface of the metal layer of the biosensor, the method further includes steps of: performing carboxylation treatment on the surface of the metal layer, and then performing carboxyl activation treatment. Specifically, the carboxylation treatment includes: contacting the metal layer with a phosphate buffer solution of mercaptopropionic acid, then washing the metal layer three times with a phosphate buffer solution and deionized water sequentially, and drying with a nitrogen gas. The carboxyl activation treatment includes: contacting the metal layer with a mixture solution of 1-(3-dimethylaminopropyl)-3-ethylcarbodiimide hydrochloride (EDC) and N-hydroxysuccinimide (NHS), then washing the metal layer three times with a phosphate buffer solution and deionized water sequentially, and drying with a nitrogen gas. The molar ratio of EDC to NHS is 4:1.

It may be understood that before performing carboxylation treatment on the surface of the metal layer, the method further includes a step of: performing hydrophilization treatment on the surface of the metal layer. Optionally, the hydrophilization treatment includes plasma etching the metal layer.

It may be understood that before perpendicularly radiating incident light onto the surface of the metal layer, the method further includes a step of: blocking free carboxyl groups on the surface of the metal layer subjected to the antibody immobilization. Specifically, the blocking free carboxyl groups includes: contacting the metal layer with a bovine serum albumin (BSA) solution, then washing the metal layer three times with a phosphate buffer solution and deionized water sequentially, and drying with a nitrogen gas.

Another embodiment of the present disclosure provides use of the biosensor in the measurement of a refractive index of a solution. Specifically, the use includes the following steps:
radiating incident light onto a surface of the metal layer in a direction perpendicular to the groove bottom of the metal groove, and measuring an initial optical parameter of reflected light;
adding dropwise different solutions with known refractive indexes onto the surface of the metal layer respectively, and measuring standard optical parameters of reflected light corresponding to the different solutions with known refractive indexes;
calculating difference values between the standard optical parameters corresponding to the different solutions with known refractive indexes and the initial optical parameter respectively, and obtaining a change relation between the optical parameter and the refractive index based on the difference values and the corresponding refractive indexes; and
adding dropwise a solution to be tested onto the surface of the metal layer, measuring an optical parameter of reflected light corresponding to the solution to be tested, calculating a difference value between the optical parameter and the initial optical parameter, and obtaining a refractive index of the solution to be tested according to the difference value and the change relation.

Preferably, a plurality of dielectric grooves are distributed in a plurality of rows and/or in a plurality of columns, the rows are parallel to each other, the columns are parallel to each other, and the rows are perpendicular to the columns. A polarization direction of the incident light is perpendicular to the row direction, or a polarization direction of the incident light is perpendicular to the column direction. For example, when the metal grooves are distributed in a two-dimensional array as shown in (b) of FIG. 20, the polarization direction of the incident light is perpendicular to the row direction, or the polarization direction of the incident light is perpendicular to the column direction.

Further, the dielectric grooves in each row are in communication with each other to form a communicated groove with a uniform width. Alternatively, the dielectric grooves in each column are in communication with each other to form a communicated groove with a uniform width. The polarization direction of the incident light is perpendicular to an extending direction of the communicated groove. For example, when the metal grooves are distributed in a one-dimensional array as shown in (a) of FIG. 20, the polarization direction of the incident light is perpendicular to an extending direction of the metal grooves.

Specific examples will be described below.

### Example 1

In this example, metal grooves of a biosensor are distributed in a one-dimensional array as shown in (a) of FIG. 20.

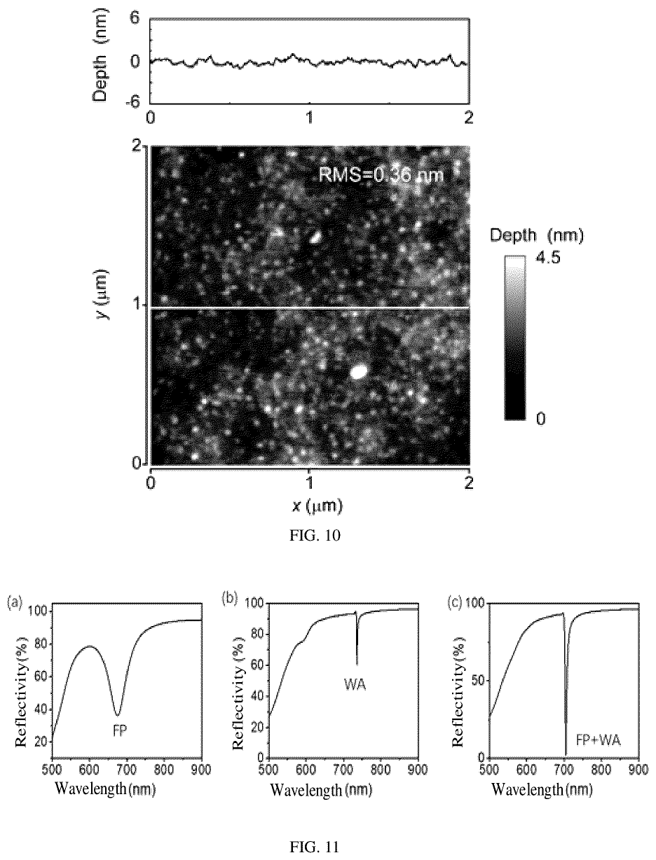

A method for preparing the biosensor in this example includes the following steps:
S101: A silicon wafer was spin-coated with a polymethyl methacrylate film, and the thickness of the polymethyl methacrylate film was controlled to be 900 nm.
S102: The polymethyl methacrylate film was subjected to electron beam lithography and development, to form a grating structure on the polymethyl methacrylate film, thereby obtaining a mask silicon wafer. A period of the grating was 700 nm, and a width of a grating stripe was 350 nm.
S103: The mask silicon wafer was subjected to reactive ion beam etching by using the polymethyl methacrylate grating structure as a mask, to form auxiliary protrusions on the silicon wafer. A groove structure was defined by two adjacent auxiliary protrusions, in which a width of a groove opening of the groove were 450 nm, a width of a groove bottom of the groove was 260 nm, a depth of the groove was 350 nm, and an array period was 700 nm.
S104: The silicon wafer obtained in S103 was etched with oxygen plasma, to remove the residual polymethyl methacrylate, thus forming a silicon template with auxiliary protrusions.
S105: The silicon template obtained in S104 was deposited with gold with a vertical thickness of 200 nm by using a magnetron sputtering instrument to obtain a gold layer. An electric current of the sputtering was 35 mA and a time period of the sputtering was 500 seconds.
S106: Liquid ultraviolet-curable epoxy resin (model: NOA61, Norland Company) was added dropwise onto the gold layer, the epoxy resin was covered with a clean glass slide, and the glass slide was pressed down firmly, so that the epoxy resin layer between the glass slide and the silicon wafer had a uniform thickness, and the gold layer was entirely covered by the epoxy resin layer. The size of the glass slide was greater than the size of the silicon template. Then, the epoxy resin was cured by irradiating from an end of the glass slide with an ultraviolet lamp. The power of the ultraviolet lamp was 48 W, and a time period of the curing was 20 minutes.
S107: The gold layer was separated from the silicon template by cutting the interface between the gold layer and the silicon template with a clean knife, thereby forming a biosensor with a smooth gold layer surface and periodical gold grooves in one-dimensional array distribution. The structural parameters of the periodical gold grooves include: a period of 700 nm, a groove depth of 350 nm, a width of a groove opening of 440 nm, a width of a groove bottom of 250 nm, and a vertical thickness of the gold layer of 200 nm. An atomic force micrograph of the gold layer is shown in FIG. 10. It can be seen from FIG. 10 that the root mean square of surface roughness of the gold layer is 0.36 nm, which indicates that the ultra-smooth gold grooves can be obtained by this method.
S108: The silicon template after separation were cleaned with acetone, ethanol, and an aqueous solution of iodine/potassium iodide sequentially to remove the residual epoxy resin and gold on the silicon template, thereby obtaining a regenerated silicon template. The ratio of iodine, potassium iodide, and deionized water in the aqueous solution of iodine/potassium iodide was 3:10:140.

The biosensor in this example has a very narrow resonance linewidth (i.e., 4.7 nm of resonance linewidth), thus having excellent sensing performance.

For verification of performance of the biosensor in this example, the parameters in this example are set as follows: w₀=440 nm, w₁=250 nm, a=700 nm, ε_{d}=1 (in the air), and εₘ is taken from experimental data of a dielectric constant of gold from Johnson-Christy. According to the formula (2), relation curves between resonance wavelengths of the FP mode with different orders and the gold groove depth were drawn, as shown by dotted lines in (a) of FIG. 14. According to the formula (1), a relation curve between the resonance wavelength of the WA mode and the gold groove depth was drawn, as shown by a dot dash line in (a) of FIG. 14. Reflection spectrum scans of gold layers with different gold groove depths were calculated by using a finite time domain difference method, as shown in a background gray map in (a) of FIG. 14, wherein different grays represent different reflectivity. By searching for intersections of dispersion curves of the two modes in (a) of FIG. 14, it is found that a dispersion curve of a resonant mode of the periodical gold grooves at the intersection is abnormally bent, occurring a reflection valley with an ultra-narrow linewidth. Specifically, when the groove depth h is 350 nm, the intensity of the reflection valley dropped to zero as shown in (b) of FIG. 14, indicating that a critical coupling condition is reached. That is, the radiation attenuation rate of the system is equal to the internal attenuation rate of the system. At this groove depth value, both the ultra-narrow resonance linewidth and the ultra-high resonance intensity (i.e., the depth of the reflection valley, defined as a difference between a maximum light intensity and a minimum light intensity at the resonance) were achieved, which indicates that the structural parameters are very ideal.

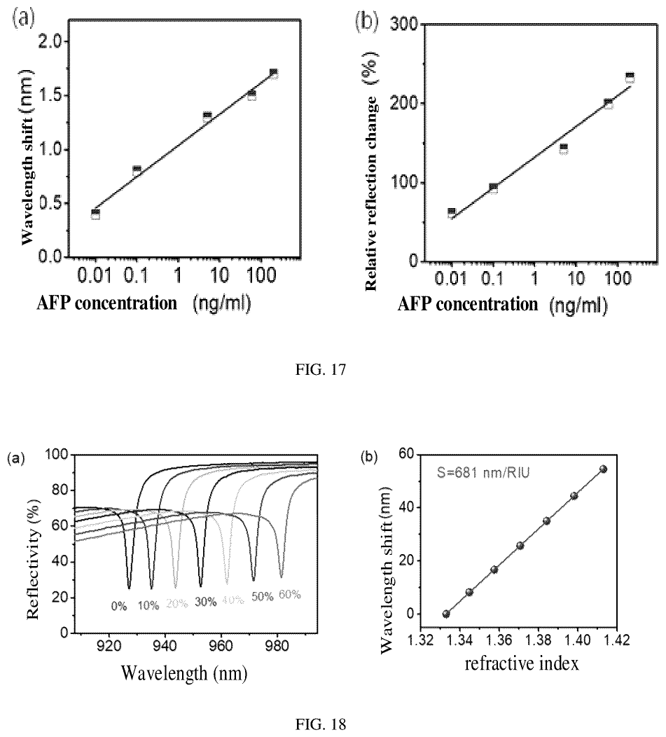

An application of the biosensor in this example in the measurement of a concentration of an antigen includes the following steps.
S201: A surface of the gold layer was subjected to hydrophilization treatment. Specifically, the biosensor obtained in S107 was etched with oxygen plasma for 30 seconds at a power of 200 W.
S202: The surface of the gold layer was subjected to carboxylation treatment. Specifically, the biosensor after treated in S201 was soaked in 10 mM phosphate buffer solution of mercaptopropionic acid for 12 hours at a room temperature, then the gold layer was washed three times with a phosphate buffer solution and deionized water sequentially, and dried with a nitrogen gas.
S203: The surface of the gold layer was subjected to carboxyl activation treatment. Specifically, the biosensor after treated in S202 was soaked in a mixed aqueous solution of 400 mM 1-(3-dimethylaminopropyl)-3-ethylcarbodiimide hydrochloride (EDC) and 100 mM N-hydroxysuccinimide (NHS) for 2 hours at a room temperature, then the gold layer was washed three times with a phosphate buffer solution and deionized water sequentially, and dried with a nitrogen gas.
S204: An antibody solution was added dropwise onto the surface of the metal layer of the biosensor and the antibody was immobilized. Specifically, 100 μg/ml of alpha-fetoprotein (AFP) antibody solution was added dropwise onto the surface of the gold layer of the biosensor after treated in S203, and the biosensor was incubated in an incubator at 37° C. for 1 hour, then the gold layer was washed three times with a phosphate buffer solution and deionized water sequentially, and dried with a nitrogen gas.
S205: The biosensor after treated in S204 was soaked in a bovine serum albumin (BSA) solution for 40 minutes, then washed three times with a phosphate buffer solution and deionized water sequentially, and dried with a nitrogen gas.
S206: Incident light was radiated onto the surface of the metal layer in a direction perpendicular to the groove bottom of the gold groove by using a microregion optical measurement system, in which a polarization direction of the incident light was perpendicular to an extending direction of the gold groove. A reflection spectrum of the biosensor in this case was recorded, as shown by solid lines in (a)-(e) in FIG. 16.
S207: AFP antigen solutions with concentrations of 0.01 ng/ml, 0.1 ng/ml, 5 ng/ml, 60 ng/ml, and 200 ng/ml were respectively added dropwise onto the gold layers of five biosensors with same structural parameters, and the biosensors were incubated in an incubator at 37° C. for 1 hour, then the gold layers were washed three times with a phosphate buffer solution and deionized water sequentially, and dried with a nitrogen gas.
S208: Incident light was perpendicularly radiated onto the gold layers of the biosensors after treated in S207 by using a microregion optical measurement system, in which an incident direction of the incident light was perpendicular to a groove bottom of the gold layer, and a polarization direction of the incident light was perpendicular to the incident direction. Reflection spectra of the biosensors in the case were recorded, as shown by dotted lines in (a)-(e) in FIG. 16.
S209: The spectra before and after the addition of the AFP antigen were compared, and a change curve between the resonance wavelength shift of the biosensor and the concentration of the AFP antigen was drawn, as shown in (a) of FIG. 17. In addition, a change curve between the relative reflectivity change of the biosensor at a wavelength of 704.645 nm and the concentration of the AFP antigen was drawn, as shown in (b) of FIG. 17. As can be seen from FIGS. 16 and 17, a measurement limit of the biosensor for the AFP antigen was 0.01 ng/ml, with a linear range from 0.01 ng/ml to 200 ng/ml. Based on the change curves in (a) and (b) of FIG. 17, the concentration of the antigen solution corresponding to the resonance wavelength shift or the relative reflectivity change of the biosensors with the same structural parameters in the linear range can be determined.

### Example 2

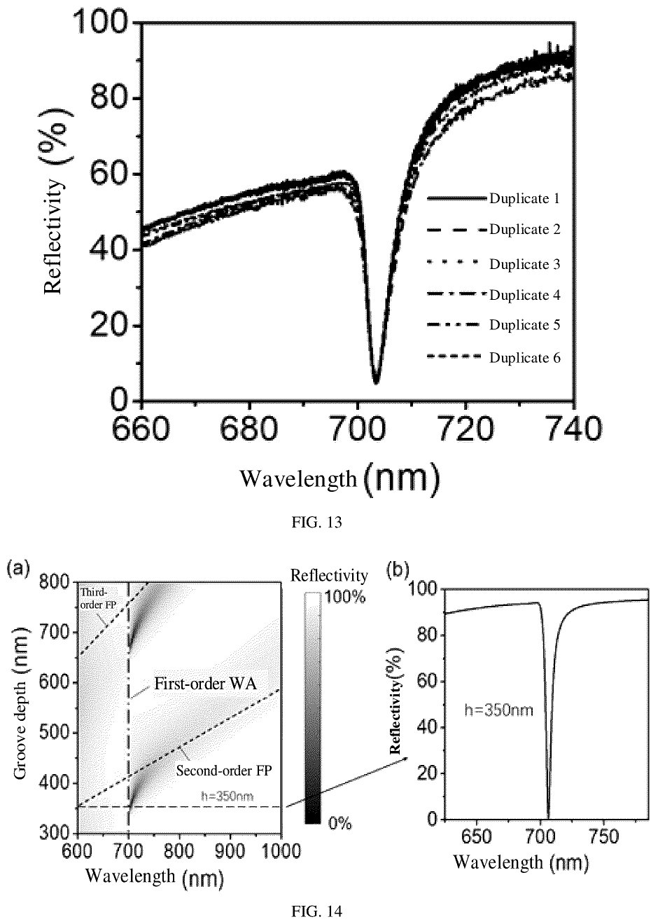

In this example, reusability of the silicon template used in the preparation method in Example 1 was verified. S105 to S108 in Example 1 were repeated, and another five biosensors prepared with the same silicon template were obtained, labeled as Duplicate 1 (i.e., the biosensor in Example 1), Duplicate 2, Duplicate 3, Duplicate 4, Duplicate 5, and Duplicate 6 respectively. Reflection spectra of the six biosensors were measured with a micro spectrometer, and results are shown in FIG. 13. As can be seen from FIG. 13, the six biosensors had almost the same spectra, in which a standard error of the resonance wavelengths was merely 0.06 nm, and a standard error of the reflected light intensity at a wavelength of 702.5 nm was merely 0.54%, which indicated that the biosensor obtained by the preparation method in Example 1 had desirable optical response consistency.

### Example 3

In this example, metal grooves of a biosensor are distributed in a one-dimensional array as shown in (a) of FIG. 20. The structural parameters of periodical gold grooves in this example are as follows: a period of 700 nm, a groove depth of 350 nm, a width of a groove opening of 450 nm, a width of a groove bottom of 260 nm, and a vertical thickness of a gold layer of 300 nm.

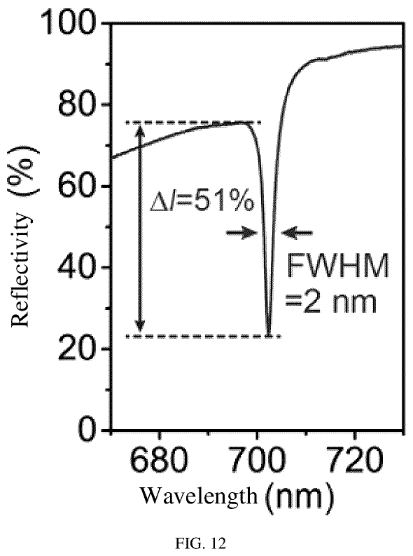

With reference to FIG. 12, a full width at half maximum of the biosensor in the coupled mode in this example was merely 2 nm. The full width at half maximum is defined as a corresponding spectral linewidth at half of a sum of light intensities at the highest point and the lowest point of a reflection valley.

A method for measuring a refractive index of glycerol in this example includes the following steps.
S301: Glycerol aqueous solutions with concentrations of 0%, 10%, 20%, 30%, 40%, 50% and 60% were respectively prepared, with corresponding refractive indexes of 1.33303, 1.34481, 1.35749, 1.3707, 1.38413, 1.39809 and 1.41299.
S302: Incident light was radiated onto a surface of a metal layer in a direction perpendicular to a groove bottom of a gold groove, in which a polarization direction of the incident light was perpendicular to an extending direction of the gold groove. An initial resonance wavelength of reflected light was measured.
S303: Glycerol aqueous solutions with different concentrations prepared in S301 were added dropwise onto the surface of the metal layer respectively, and the resonance wavelength of corresponding reflected light was measured. It may be understood that after each measurement, the biosensor was cleaned with deionized water.
S304: Difference values between resonance wavelengths of reflected light corresponding to the solutions with different refractive indexes and the initial resonance wavelength were calculated respectively, and a change curve between the resonance wavelength difference and the refractive index was obtained based on the difference values and corresponding refractive indexes, as shown in (b) of FIG. 18.
S305: A solution to be tested was added dropwise onto the surface of the metal layer, a resonance wavelength of reflected light corresponding to the solution to be tested was measured, a difference value between the resonance wavelength and the initial resonance wavelength was calculated, and a refractive index of the solution to be tested was obtained according to the difference value and the change curve as shown in (b) of FIG. 18. In S303, in the presence of glycerol aqueous solutions with different concentrations, the relation between the reflectivity of the reflected light and the resonance wavelength can also be obtained, as shown in (a) of FIG. 18. By combining (a) and (b) of FIG. 18, it can be calculated that the biosensor has a sensitivity (S) of 681 nm/RIU and a wavelength quality factor (FOM_{λ}) up to 285. S=Δλ/Δn, Δλ represents a change value of the wavelength, and An represents a change value of the refractive index. FOM_{λ}=S/FWHM, FWHM represents a full width at half maximum of resonance.

### Example 4

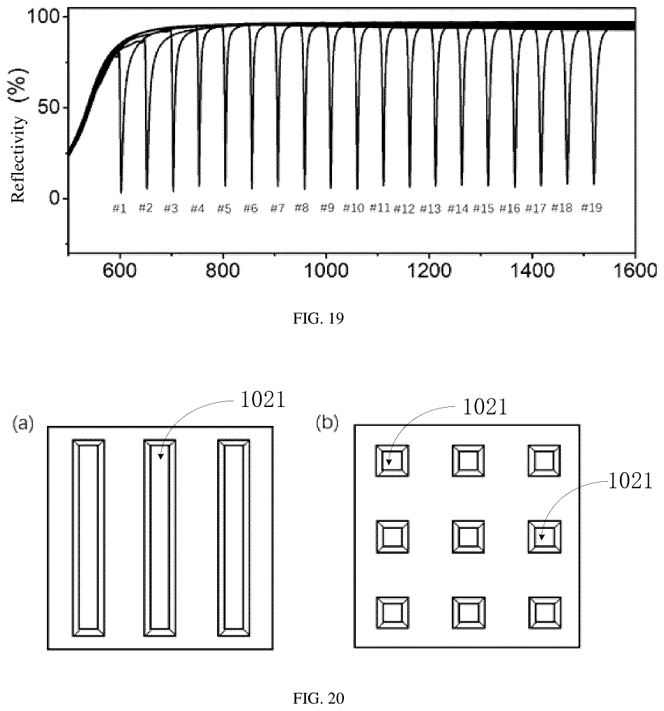

In this example, preferred sizes of a gold groove under different working wavelengths were designed. In this example, metal grooves of a biosensor are distributed in a one-dimensional array, as shown in (a) of FIG. 20.
S401: A period of the gold groove was fixed to be 600 nm, a width of a groove opening was 420 nm, and a width of a groove bottom was 220 nm. A resonance wavelength of a first-order WA mode was determined to be 600 nm according to the formula (1). According to the formula (2), a groove depth range of a second-order FP mode at a resonance wavelength around 600 nm was estimated to be from 200 nm to 400 nm. Reflection spectra of gold grooves with different groove depths from 200 nm to 400 nm were simulated by using a finite time domain difference method, and a FP-WA coupled mode with the narrowest linewidth and the deepest reflection valley was searched, as shown in #1 of FIG. 19.
S402: S401 was repeated, in which the period of the gold groove was fixed to be from 600 nm to 1500 nm. Reflection spectra within the groove depth range corresponding to different periods were scanned by using the finite time domain difference method. A FP-WA coupled mode with the narrowest linewidth was searched. Finally, optimal structural parameters of several gold nano-groove arrays under different working wavelengths were obtained, as shown in the following table. The reflection spectra of the gold grooves under different working wavelengths are shown in FIG. 19.

| | | | Width of | Width of | Thickness | | |
|---|---|---|---|---|---|---|---|
| | | Depth of | groove | groove | of gold | Working | Resonance |
| Serial | Period | groove | opening | bottom | layer | wavelength | linewidth |
| No. | (nm) | (nm) | (nm) | (nm) | (nm) | (nm) | (nm) |
| #1 | 600 | 300 | 420 | 220 | 200 | 602.1 | 6.9 |
| #2 | 650 | 330 | 440 | 240 | 200 | 652.2 | 6.1 |
| #3 | 700 | 350 | 440 | 240 | 200 | 703.3 | 4.5 |
| #4 | 750 | 375 | 450 | 250 | 200 | 753.6 | 3.5 |
| #5 | 800 | 400 | 460 | 260 | 200 | 804.6 | 3.4 |
| #6 | 850 | 425 | 460 | 260 | 200 | 856.0 | 3.5 |
| #7 | 900 | 450 | 480 | 480 | 200 | 906.5 | 3.9 |
| #8 | 950 | 475 | 480 | 280 | 200 | 958.3 | 4.2 |
| #9 | 1000 | 505 | 490 | 290 | 200 | 1009 | 4.3 |
| #10 | 1050 | 530 | 490 | 290 | 200 | 1060.6 | 4.5 |
| #11 | 1100 | 555 | 490 | 290 | 200 | 1111.3 | 4.5 |
| #12 | 1150 | 580 | 500 | 300 | 200 | 1162.3 | 4.8 |
| #13 | 1200 | 610 | 520 | 320 | 200 | 1212.5 | 5.1 |
| #14 | 1250 | 640 | 530 | 330 | 200 | 1263.5 | 5.5 |
| #15 | 1300 | 665 | 530 | 330 | 200 | 1314.6 | 5.8 |
| #16 | 1350 | 695 | 540 | 340 | 200 | 1366.3 | 6.4 |
| #17 | 1400 | 725 | 570 | 370 | 200 | 1417.1 | 7.1 |
| #18 | 1450 | 755 | 580 | 380 | 200 | 1468.1 | 7.8 |
| #19 | 1500 | 785 | 590 | 390 | 200 | 1519.7 | 8.3 |

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims, and the description and the accompanying drawings can be used to explain contents of the claims.

1. A biosensor for implementing a FP-WA coupled mode, the biosensor comprising:
a dielectric layer; and
a metal layer;
wherein the dielectric layer comprises a plurality of dielectric grooves, the plurality of dielectric grooves are periodically distributed at equal intervals, and an opening width of the dielectric groove gradually decreases in a direction from a groove opening to a groove bottom; and the metal layer is disposed on the dielectric layer and comprises metal grooves that are in one-to-one correspondence with the dielectric grooves;
a period of the metal groove satisfies formula (1):
$${λ}_{W A} = \frac{a}{i} \sqrt{{ε}_{d}} ; \qquad Formula ( 1 )$$
a depth of the metal groove, a width of the groove opening of the metal groove, and a width of the groove bottom of the metal groove satisfy formula (2):
$$h = \frac{2 π m - {φ}_{r}}{2} \cdot \frac{{w}_{0} - {w}_{1}}{k} \{ \frac{1}{- {w}_{1} \sqrt{1 + \frac{A}{{w}_{1}}} + {w}_{0} \sqrt{1 + \frac{A}{{w}_{0}}} + \frac{A}{2} ln [ ( \frac{\sqrt{1 + \frac{A}{{w}_{1}}} - 1}{\sqrt{1 + \frac{A}{{w}_{1}}} + 1} ) ( \frac{\sqrt{1 + \frac{A}{{w}_{0}}} + 1}{\sqrt{1 + \frac{A}{{w}_{0}}} - 1} ) ]} \} ; \qquad Formula ( 2 )$$
$$wherein A = \frac{2 \sqrt{{ε}_{d} ( {ε}_{d} - {ε}_{m} )}}{- {ε}_{m} k} , k = \frac{2 π \sqrt{{ε}_{d}}}{{λ}_{WG}} ;$$
and
a represents the period of the metal groove, h represents the depth of the metal groove, w₀ represents the width of the groove opening of the metal groove, w₁ represents the width of the groove bottom of the metal groove, i represents an order of a WA mode, λ_{WA} represents a resonance wavelength of the WA mode, m represents an order of an FP mode, λ_{WG} represents a resonance wavelength of the FP mode, k represents a resonance wave number of the FP mode, φᵣ represents a sum of reflection phases of the FP mode at the groove opening of the metal groove and the groove bottom of the metal groove, ε_{d} represents a dielectric constant of an environment where the biosensor is located, and εₘ represents a dielectric constant of the metal layer.

2. The biosensor according to claim 1, wherein the period of the metal groove is from 600 nm to 1500 nm, the depth of the metal groove is from 300 nm to 800 nm, the width of the groove opening of the metal groove is from 400 nm to 600 nm, and the width of the groove bottom of the metal groove is from 200 nm to 400 nm.

3. The biosensor according to claim 1, wherein a resonance linewidth of the biosensor is from 3 nm to 9 nm; and/or
a thickness of the metal layer is from 200 nm to 500 nm; and/or
a root mean square of surface roughness of the metal layer is from 0.2 nm to 1.9 nm.

4. The biosensor according to claim 1, wherein the plurality of dielectric grooves are distributed in a plurality of rows and/or in a plurality of columns, the rows are parallel to each other, the columns are parallel to each other, and the rows are perpendicular to the columns.

5. The biosensor according to claim 4, wherein the dielectric grooves in each row are in communication with each other to form a communicated groove with a uniform width, or the dielectric grooves in each column are in communication with each other to form a communicated groove with a uniform width.

6. A method for preparing the biosensor according to claim 1, comprising:
forming auxiliary protrusions that are distributed periodically at equal intervals on a template;
forming a metal layer on a surface of the template, the metal layer comprising metal grooves that are in one-to-one correspondence with the auxiliary protrusions;
forming a dielectric layer on the metal layer; and
separating the metal layer from the template.

7. A method for measuring a parameter of interest of a target, comprising using the biosensor according to claim 1 to measure the parameter of interest of the target.

8. The n according to claim 7, comprising:
radiating incident light onto a surface of the metal layer in a direction perpendicular to the groove bottom of the metal groove, and measuring an initial optical parameter of reflected light;
adding dropwise different target solutions with known parameters of interest onto the surface of the metal layer respectively, and measuring standard optical parameters of reflected light corresponding to the target solutions;
calculating difference values between the standard optical parameters corresponding to the different target solutions with known parameters of interest and the initial optical parameter respectively, and obtaining a change relation between the optical parameter and the parameter of interest of the target solution based on the difference values and the corresponding known parameters of interest; and
adding dropwise a solution to be tested onto the surface of the metal layer, measuring a sample optical parameter of reflected light corresponding to the solution to be tested, calculating a difference value between the sample optical parameter and the initial optical parameter, and obtaining a parameter of interest of the solution to be tested according to the difference value and the change relation.

9. The method according to claim 8, wherein a plurality of dielectric grooves are distributed in a plurality of rows and/or in a plurality of columns, the rows are parallel to each other, the columns are parallel to each other, and the rows are perpendicular to the columns; and a polarization direction of the incident light is perpendicular to a row direction, or a polarization direction of the incident light is perpendicular to a column direction.

10. The method according to claim 9, wherein the dielectric grooves in each row are in communication with each other to form a communicated groove with a uniform width, or the dielectric grooves in each column are in communication with each other to form a communicated groove with a uniform width, and the polarization direction of the incident light is perpendicular to an extending direction of the communicated groove.

11. The method according to claim 7, wherein the method is for a non-disease diagnosis purpose.
